(19) United States
(12) Patent Application Publication
(10) Pub. No.: US 2009/0181455 A1
(43) Pub. Date: Jul. 16, 2009

(54) Modulation of gene expression using insulator binding proteins

(75) Inventors: Alan P. Wolffe, Orinda, CA (US); Elizabeth J. Wolffe, Orinda, CA (US)

Correspondence Address: ROBINS & PASTERNAK, 1731 EMBARCADERO ROAD, SUITE 230, PALO ALTO, CA, 94303, US

(73) Assignee: Sangamo BioSciences, Inc.

(21) Appl. No.: 12/287,409

(22) Filed: Oct. 9, 2008

Related U.S. Application Data
(62) Division of application No. 10/446,901, filed on May 27, 2003.
Application No. 10/446,901 is a continuation of application No. PCT/US01/44654, filed on Nov. 28, 2001.
(60) Provisional application No. 60/253,678, filed on Nov. 28, 2000.

(51) Int. Cl. C12N 5/08; C12N 5/04; C12N 5/06

(52) U.S. Cl. 435/366; 435/410; 435/325

(57) ABSTRACT

Methods and compositions for regulating gene expression are provided. In particular, methods and compositions including insulator domains for targeted regulation of a gene or transgene are provided.

### CROSS-REFERENCE TO RELATED APPLICATIONS

This application is a divisional of U.S. patent application Ser. No. 10/446,901, filed May 27, 2003, which is a continuation of PCT/US01/44654, filed Nov. 28, 2001, and claims the benefit of provisional application 60/253,678, filed Nov. 28, 2000, all of which applications are hereby incorporated by reference in their entireties.

### TECHNICAL FIELD

This disclosure is in the field of molecular biology and medicine. More specifically, it relates to modulation of gene expression using functional domains derived from insulator binding proteins and functional fragments thereof.

### BACKGROUND

The organization of cellular DNA plays a crucial role in the regulation of gene expression. Cellular DNA generally exists in the form of chromatin, a complex comprising nucleic acid and protein. Indeed, most cellular RNAs also exist in the form of nucleoprotein complexes. The nucleoprotein structure of chromatin has been the subject of extensive research, as is known to those of skill in the art. In general, chromosomal DNA is packaged into nucleosomes. A nucleosome comprises a core and a linker. The nucleosome core comprises an octamer of core histones (two each of H2A, H2B, H3 and H4) around which is wrapped approximately 150 base pairs of chromosomal DNA. In addition, a linker DNA segment of approximately 50 base pairs is associated with linker histone H1. Nucleosomes are organized into a higher-order chromatin fiber and chromatin fibers are organized into chromosomes. See, for example, Wolffe “Chromatin: Structure and Function” 3^{rd} Ed., Academic Press, San Diego, 1998.

Further, cellular chromatin, including nucleosome structure, is organized into a higher order structure of regions or “domains.” In those tissues where a given gene or gene cluster is active, the domain is sensitive to DNase I, suggesting that the chromatin of an active domain is in a loose, decondensed configuration that is easily accessible to trans-acting factors (Lawson et al. (1982). J. Biol. Chem., 257:1501-1507; Groudine et al. (1983). Proc, Natl. Acad. Sci. USA, 80:7551-7555). By contrast, in those tissues where the same gene is not active, the chromatin of the domain is in a tight configuration that is inaccessible to transacting factors. Thus, decondensing the higher order chromatin structure of a domain is required before regulatory factors (e.g., transcription factors that bind to specific DNA sequences) can interact with target sequences, thereby determining the transcriptional competence of that domain.

The higher order chromatin structure of genes, as well as the flanking region surrounding the genes, are uniform throughout each domain, but are discontinuous in the regions, loosely termed “boundaries”, between adjacent domains (Eissenberg, et al. (1991) TIG 7:335-340). It is generally thought that domains are delimited by special nucleoprotein structures assembled at specific sites along the eukaryotic chromosome. The specialized chromosomal regions, termed insulators, are thought to be associated with the boundaries of repressive or active domains. Insulator elements have been defined by two characteristic effects on gene expression: (1) they confer position-independent transcription to transgenes stably integrated into the chromosome (Bonifer et al. (1990) EMBO J. 9:2843-2848; Kellum et al. (1991) Cell 64:941-950) and (2) they buffer a promoter from activation by enhancers when located between the two (Kellum et al. (1992) Mol. Cell. Biol. 12:2424-2431; Chun et al. (1993) Cell 74:505-514). Thus, insulator elements prevent the transmission of chromatin structural features associated with repressive or active domains of chromatin.

Gene expression of cellular DNA is also regulated by DNA methylation of CpG dinucleotides. DNA methylation is required for normal development (Ohki et al (1999) EMBO J. 18:6653-6661; Okano et al. (1999) Cell 99:247-257); is correlated with genomic imprinting (Ashburner (1972) Results Probl Cell Differ 4:101-151; Grunstein et al. (1997) Nature 389:349-352) and X-chromosome inactivation (Heard et al. (1997) Annual Rev Genet. 31:571-610). A large body of evidence indicates that cytosine methylation leads to the assembly of a specialized, heritable, repressive chromatin architecture through the recruitment of histone deacetylases (Bird and Wolffe (1999) Cell 99:451-454; Siegfried et al. (1997) Curr Biol 7:R305-307). However, the precise role of DNA methylation in tissue specific regulation of imprinted and non-imprinted genes remains contentious (Bird (1997) Trends Genet. 13:469-472).

A DNA binding protein containing 11 zinc fingers, termed CTCF (for CCCTC-binding factor), has been shown to bind to certain known vertebrate insulator elements (Bell et al. (1999) Cell 98:387-396). CTCF is an abundant, highly-conserved protein. (Klenova et al. (1993) Mol. Cell. Biol. 13:7612-7624; Fillippova et al. (1996) Mol. Cell. Biol. 16:2808-2813); Burcin et al. (1997) Mol. Cell. Biol. 17:1218-1288). The zinc finger domain of CTCF binds preferentially to regions of DNA with high GC nucleotide content, for example in the chicken c-myc gene each of the 50 base pair long CTCF binding sites contains 65-87% GC.

Further, CTCF also appears to recognize the 21 base pair CpG-rich sequence repeats located within a 2 kb “imprinting control region” that lies between the insulin-like growth factor II (Igf2) and H19 genes (Bell et al. (2000) Nature 405:482-485). Igf2-H19 represents the most extensively studied example of the phenomenon termed genomic imprinting (genes that inherit gametic markers that establish parent of origin-dependent expression patterns in the soma). The Igf2 and H19 genes are expressed mono-allelically from opposite parental alleles (with Igf2 being expressed from the paternal, and H19 form the maternal chromosome) and are members of a cluster of imprinted loci at the distal part of chromosome 7 (Bartolomei et al. (1997) Nature 351:153-155; DeChiara et al. (1991) Cell 64:849-859; Horsthemke et al (1999) in Genomic Imprinting: An Interdisciplinary Approach, R. Ohlsson ed.) vol 25, pp. 91-118 (Springer-Verlag, Berlin). The imprinting control region of the Igf2-H19 locus is differentially methylated between paternal and maternal chromosomes. (Elson et al. (1997) Mol. Cell. Biol. 17:309-317), and binding of CTCF to its recognition sequences in the imprinting control region is sensitive to CpG methylation of these sequences. When the imprinting control region is unmethylated (as found on maternal chromosomes), CTCF binds to the insulator element between the two genes, preventing an enhancer which lies distal to the H19 gene from acting on the Igf2 promoter. Thus, the H19 gene is active and the Igf2 gene is inactive on the maternal chromosome. Conversely, when the imprinting control region and the H19 gene are methylated (as found on paternal chromosomes), CTCF fails to bind to the insulator. (Hark et al. (2000) Nature 405:486; Chung et al. (1993) Cell 74:505-514). In this case, the enhancer distal to the H19 gene activates the Igf2 promoter, but methylation of the imprinting control region prevents transcription of the H19 gene, even in the presence of its enhancer. Thus, on the paternal chromosome, the Igf2 gene is active, and the H19 gene is inactive

Based on these and other results, the following picture of insulators, their function and their mechanism of action has emerged. Insulators are sequences which define boundaries between chromosomal domains, thereby acting as a barrier to the influence of one chromosomal domain upon another. Their two most well-characterized functions of insulators are to block the transmission of repressive influences from one chromosomal domain to another (e.g., prevention of position effects) and to inhibit the activating effect of an enhancer upon a promoter, when interposed therebetween. Insulators are able to carry out these functions by serving as binding sites for insulator binding proteins, which are likely to assemble protein complexes onto the insulator sequence. As one example, sequences such as the Igf2-H19 imprinting control region function as binding sites for proteins such as CTCF, which function to block enhancer action. An example of the ability of insulator sequences to blocking repression of a gene by complexes which repress gene expression in an adjacent chromosomal domain is provided by Corces et al. (1997) in Nuclear Organization, Chromatin Structure and Gene Expression (van Driel, R. and Otte, A. P., eds.) pp. 83-98, Oxford University Press, Oxford; Udvardy (1999) EMBO J. 18:1-8. For a general review of insulators, their function and their mechanism of action, see Bell et al. (1999) Curr. Opin. Genet. Devel. 9:191-198 and references cited therein.

Currently, the ability of an insulator binding protein to demarcate a chromosomal domain is limited to those regions of a chromosome that have sufficient proximity to insulator sequences. It would be useful to be able to target the activity of insulator binding proteins, such that a unique chromosomal architecture could be established at any predetermined region of the chromosome.

### SUMMARY

The compositions and methods described herein allow for targeting of insulator binding proteins to establish unique chromosomal domains at predetermined regions of the chromosome. It is demonstrated herein that insulator binding proteins interact with a diverse spectrum of variant target sites and that these proteins contain multiple components that cooperate to confer their unique properties. In view of the novel observations described herein, specifically targeted regulatory molecules containing a DNA-binding domain and an insulator domain can be designed. These molecules can insulate transgenes and other exogenous polynucleotides from silencing in order to obtain sustained expression of such genes. In addition, the molecules can be used to specifically target genes for silencing, for example by interfering with enhancer function by targeting a DNA-binding protein-insulator domain fusion molecule between an enhancer and a promoter.

Thus, in one aspect, a method of modulating expression of a gene, the method comprising the step of contacting a region of DNA in cellular chromatin with a fusion molecule that binds to a binding site in cellular chromatin, wherein the fusion molecule comprises a DNA binding domain or functional fragment thereof and an insulator domain or functional fragment thereof is provided. In various embodiments, the DNA-binding domain of the fusion molecule comprises a zinc finger DNA-binding domain. Further, the DNA binding domain binds to a target site in a gene encoding a product selected from the group consisting of vascular endothelial growth factor, erythropoietin, androgen receptor, PPAR-γ2, p16, p53, pRb, dystrophin and e-cadherin. In other embodiments, the insulator domain is derived from, for example, a CTCF polypeptide; a su(Hw) polypeptide or a polycomb group protein. Further, the gene can be, for example, in a plant cell or an animal cell (e.g., a human cell). In certain embodiments, the fusion molecule is a polypeptide. In various embodiments, the modulation comprises repression of expression of the gene. In other embodiments, the modulation comprises activation of expression of the gene. Further, in certain embodiments, the binding site is between an enhancer and a promoter and further wherein binding of the fusion molecule interferes with the function of the enhancer. In certain other embodiments, the target gene is a transgene and the modulation comprises activation or repression of the transgene.

In any of the methods described herein, the fusion molecule can be a fusion polypeptide and the method can further comprise the step of contacting the cell with a polynucleotide encoding the fusion polypeptide, wherein the fusion polypeptide is expressed in the cell. Further, in any of the methods described herein a plurality of fusion molecules (e.g., one or more zinc finger DNA-binding domain proteins) can be contacted with cellular chromatin, wherein each of the fusion molecules binds to a distinct binding site. Preferably, the expression of a plurality of genes is modulated. The cellular chromatin can be, for example, a plant cell or an animal cell (e.g., a human cell).

In other aspects, a fusion polypeptide comprising: (a) an insulator domain or functional fragment thereof; and (b) a DNA binding domain or a functional fragment thereof is described. In certain embodiments, the DNA-binding domain is a zinc finger DNA binding domain and/or the insulator domain is, for example, CTCF, su(Hw) or polycomb group proteins. In certain embodiments, the DNA-binding domain binds to a target site in a gene encoding a product selected from the group consisting of vascular endothelial growth factor, erythropoietin, androgen receptor, PPAR-γ2, p16, p53, pRb, dystrophin and e-cadherin.

In other aspects, a polynucleotide encoding any of the fusion polypeptides described herein is provided.

In yet other aspects, a host cell comprising any of the fusion polypeptides or polynucleotides described herein is provided.

In still further aspects, described herein is a method of altering the chromatin structure of a gene, the method comprising the step of contacting a region of DNA in cellular chromatin with a fusion molecule that binds to a binding site in cellular chromatin, wherein the fusion molecule comprises a DNA binding domain or functional fragment thereof and an insulator domain or functional fragment thereof.

As will become apparent, preferred features and characteristics of the aspects described herein are applicable to any other aspects.

### BRIEF DESCRIPTION OF THE DRAWINGS

FIG. 1A is a schematic depiction of the mouse Igf2-H19 genomic region. The upper line shows the locations of the Igd2 and H19 genes and their regulatory elements, including the differentially methylated domain (DMD) and the enhancers. The middle line shows an expanded view of the DMD, numbered with respect to the H19 transcriptional start site. Below is shown the locations of fragments of the DMD that were 5′ end-labeled and used for binding analysis. Ten fragments, each approximately 200-bp-long, covered the following regions: (1) from −3081 to −2876; (2) from −2947 to −2763; (3) from −2808 to −2635; (4) from −2690 to −2499; (5) from −2553 to −2399; (6) from −2355 to −2227; (7) from −2284 to −2095; (8) from −2164 to −1 945; (9) from −1995 to −1 831; (10) from −1 834 to −1 579. FIG. 1B shows gel-shift assays to test for binding of the 11 zinc finger (ZF) CTCF domain synthesized from the pCITE4a-1 1 ZF vector with the DMD1 to DMD10 DNA fragments. Lanes 1, 2, and 3 of each panel correspond to gel-shift reactions with no protein, with the negative luciferase protein control, and the 11 ZF protein, respectively. Fragments producing shifted complexes are indicated on gel sides by arrowheads.

FIG. 2A shows DNAse I footprinting results from the DMD4 and DMD7 regions using CTCF-binding sequences. “G” refers to the Maxam-Gilbert sequencing G ladders and “F and B” refer to free and CTCF-bound DNA probes, respectively. “FP” refers to footprint regions protected from nuclease attack and “HS” refers to DNaseI hypersensitive sites induced upon CTCF binding. FIG. 2B shows results of DMS-methylation interference assays, carried out with full-length CTCF. The guanines that cannot be modified by DMS without losing contact with CTCF, are shown by bars on the sides of the sequencing gel images. FIG. 2C summarizes the results of the footprinting and methylation assays. Portions of the nucleotide sequences of DMD4 and DMD7 are shown with critical contact G-residues indicated by filled squares (on each strand). DNA sequences protected by CTCF from DNAseI digestion are underlined or overlined. The CpG pairs (BstUI sites), that include dGs critical for CTCF recognition, are indicated by arrowheads. FIG. 2D is a schematic depicting localization of the CTCF binding sites on the chromatin map of the maternally derived H19 DMD allele. The locations of the DNase footprints on the DMD 4 and DMD 7 fragments are indicated above the line. Rectangles along the line depict estimated nucleosome positions on the maternal allele. The vertical bars identify CpG dinucleotides. Below the line, the 21 bp conserved repeats are indicated by vertical rectangles, and the locations of NHSSs (generated by DNase I and micrococcal nuclease (MNase) are shown as arrows. The numbers indicate nucleotide positions relative to the +1 transcriptional start site of the H19 gene.

FIG. 3A shows that there is virtually complete methylation of CpGs at the BstUI sites within the CTCF-binding core sequences identified in FIG. 2C. Control (unmethylated) and Sss I methylase-treated DMD4 and DMD7 fragments were 5′-end-labelled, incubated with the BstUI methylation-sensitive restriction enzyme, and analyzed by polyacrylamide gel electrophoresis followed by autoradiography. Only control fragments are digested by BstUI (Lanes 3). FIGS. 3B and 3C show electrophoretic mobility shift assays, for binding of control unmethylated (lanes “cont”) or Sss1-methylated (lanes “Sss1”) DMD4 and DMD7 DNA fragments to increasing amounts of CTCF as indicated at the top of each panel. Free (F) and CTCF-bound (B) probes are indicated. FIG. 3D is a gel shift assay showing preferred binding of CTCF to an unmethylated binding site in a mixture of methylated and umethylated binding sites. Lanes 1 and 2 contain equal amounts of methylated DMD7 probe and unmethylated DMD4 probes, while lane 3 contains a mixture of unmethylated DMD 4 and unmethylated DMD7. Lanes 2 and 3 contain CTCF; lane 1 contains no protein. In FIG. 3E depicts a reciprocal experiment to that shown in FIG. 3D. Lanes 1 and 2 contain equal amounts of methylated DMD4 fragment and unmethylated DMD7 fragment as control, lane 3 contains a mixture of unmethylated DMD4 and DMD7. Lanes 2 and 3 contain CTCF; lane 1 contains no protein. In FIGS. 3D and 3E, filled arrowheads indicate the position of a CTCF-DMD4 complex, that can be distinguished from that of CTCF-DMD7 complex (open arrowheads) due to the difference in mobility induced by DNA bending that occurs upon CTCF binding. Thus, CTCF binding to both DMD4 and DMD7 sites is CpG-methylation sensitive.

FIG. 4A presents the results of an electrophoretic mobility shift assay, showing that specific sequence changes within the DMD destroy the CTCF recognition elements. F indicates free probe and B indicates CTCF-bound probe. The location of the probe fragment within the H19 5′-flanking region is shown below the autoradiogram. Numbering is with respect to the H19 transcriptional start site. FIG. 4B shows H19 minigene expression, as determined by RNase protection of RNA extracted from JEG-3 cells which were maintained for 9 days following transfection with episomal vectors. GAP (Glyceraldehyde 3-phosphate dehydrogenase) mRNA signal is diagnostic for input RNA levels. Schematic maps of the various constructs used in this study are also shown below the autoradiogram of the gel. The maps, which are to scale, do not show the entire PREP vector. “DMD” refers to the H19 differentially methylated domain. All other symbols are indicated in the panel. FIG. 4C is a graph depicting H19 minigene expression in transfected JEG-3 cells as quantitated both with respect to RNA input and episome copy number. The SV40 enhancer-driven expression of the pREPH19A construct was assigned a value of 100 and the value for all other samples was determined related to this value. The mean deviation of minimally three different experiments is indicated for each vector construct (unless the differences were too small to allow visualization).

FIG. 5 are gels depicting parent of origin-specific association of CTCF with the chromatin of the H19 5′-flank. Formaldehyde-cross-linked DNA was derived from fetal liver of reciprocal intraspecific hybrid crosses of M. m. domesticus and M. m. musculus and was immunopurified with an antibody to CTCF, followed by PCR-amplification. The PCR primers spanned a polymorphic Bsm Al site situated in the 5′-end of the H19 DMD and were specific for the M. m. domesticus allele.

### DETAILED DESCRIPTION

Disclosed herein are compositions containing insulator domains or functional fragments thereof, and methods of preparing and using these compositions. The methods and compositions allow for targeted modulation of expression of a target gene.

Insulators are cis-acting elements located at or near the junctions between chromatin domains. Certain DNA binding proteins such as, for example, CTCF, have been shown to exhibit specificity for these cis elements. It is now described herein that CTCF interacts with a diverse spectrum of targets sites, that binding of CTCF to at least some of its target sites is sensitive to methylation of the target sequence, and that methylation-sensitive binding of CTCF to an insulator sequence is involved in establishing parent of origin-dependent expression of imprinted genes. Thus, CTCF is an example of a versatile, multivalent insulator-binding protein which is both structurally and functionally involved in regulation of gene expression.

Thus, the methods and compositions disclosed herein allow for modulation of gene expression by employing a composition comprising an insulator-binding protein domain (“insulator domain”) or functional fragment thereof. The insulator domains are selected for their ability to affect transcription, for example for their capacity to interact with methylated sites and/or facilitate modulation of enhancer/promoter functions.

Accordingly, compositions and methods useful in modulating expression of a target gene are provided. Provided herein are compositions and methods useful in sustaining expression of a transgene by, for example, blocking position effect-dependent repression or, alternatively, for silencing genes by interfering with enhancer functions. The compositions typically comprise a fusion molecule comprising an insulator domain and a DNA-binding domain. In one preferred embodiment, the DNA binding domain comprises a zinc finger DNA-binding domain, also known as a zinc finger protein (ZFP). In certain embodiments, the DNA-binding portion of the insulator binding protein is not present in the fusion molecule. Fusion molecules such as these can be used for targeting the function of the insulator domain to a predetermined region of a chromosome.

Thus, it will be apparent to one of skill in the art that insulator domains or functional fragments thereof facilitate the regulation of many processes involving gene expression including, but not limited to, replication, recombination, repair, transcription, telomere function and maintenance, sister chromatid cohesion, mitotic chromosome segregation, binding of transcription factors and propagation and/or maintenance of chromatin structural features related to transcriptional activation and repression.

General

Use of the disclosed compositions and practice of the disclosed methods employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

The terms “nucleic acid,” “polynucleotide,” and “oligonucleotide” are used interchangeably and refer to a deoxyribonucleotide or ribonucleotide polymer in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity; i.e., an analogue of A will base-pair with T.

Chromatin is the nucleoprotein structure comprising the cellular genome. “Cellular chromatin” comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

An “exogenous molecule” is a molecule that is not normally present in a cell, but can be introduced into a cell by one or more genetic, biochemical or other methods. Normal presence in the cell is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotien, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., protein or nucleic acid (i.e., an exogenous gene), providing it has a sequence that is different from an endogenous molecule. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous molecule” is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and components of chromatin remodeling complexes.

A “fusion molecule” is a molecule in which two or more subunit molecules are linked, preferably covalently. The subunit molecules can be the same chemical type of molecule, or can be different chemical types of molecules. Examples of the first type of fusion molecule include, but are not limited to, fusion polypeptides (for example, a fusion between a ZFP DNA-binding domain and an insulator domain) and fusion nucleic acids (for example, a nucleic acid encoding the fusion polypeptide described supra). Examples of the second type of fusion molecule include, but are not limited to, a fusion between a triplex-forming nucleic acid and a polypeptide, and a fusion between a minor groove binder and a nucleic acid.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Gene activation” and “augmentation of gene expression” refer to any process which results in an increase in production of a gene product. A gene product can be either RNA (including, but not limited to, mRNA, rRNA, tRNA, and structural RNA) or protein. Accordingly, gene activation includes those processes which increase transcription of a gene and/or translation of a mRNA. Examples of gene activation processes which increase transcription include, but are not limited to, those which facilitate formation of a transcription initiation complex, those which increase transcription initiation rate, those which increase transcription elongation rate, those which increase processivity of transcription and those which relieve transcriptional repression (by, for example, blocking the binding of a transcriptional repressor). Gene activation can constitute, for example, inhibition of repression as well as stimulation of expression above an existing level. Examples of gene activation processes which increase translation include those which increase translational initiation, those which increase translational elongation and those which increase mRNA stability. In general, gene activation comprises any detectable increase in the production of a gene product, preferably an increase in production of a gene product by about 2-fold, more preferably from about 2- to about 5-fold or any integer therebetween, more preferably between about 5- and about 10-fold or any integer therebetween, more preferably between about 10- and about 20-fold or any integer therebetween, still more preferably between about 20- and about 50-fold or any integer therebetween, more preferably between about 50- and about 100-fold or any integer therebetween, more preferably 100-fold or more.

“Gene repression” and “inhibition of gene expression” refer to any process which results in a decrease in production of a gene product. A gene product can be either RNA (including, but not limited to, mRNA, rRNA, tRNA, and structural RNA) or protein. Accordingly, gene repression includes those processes which decrease transcription of a gene and/or translation of a mRNA. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Examples of gene repression processes which decrease translation include those which decrease translational initiation, those which decrease translational elongation and those which decrease mRNA stability. Transcriptional repression includes both reversible and irreversible inactivation of gene transcription. In general, gene repression comprises any detectable decrease in the production of a gene product, preferably a decrease in production of a gene product by about 2-fold, more preferably from about 2- to about 5-fold or any integer therebetween, more preferably between about 5- and about 10-fold or any integer therebetween, more preferably between about 10- and about 20-fold or any integer therebetween, still more preferably between about 20- and about 50-fold or any integer therebetween, more preferably between about 50- and about 100-fold or any integer therebetween, more preferably 100-fold or more. Most preferably, gene repression results in complete inhibition of gene expression, such that no gene product is detectable.

“Eucaryotic cells” include, but are not limited to, fungal cells (such as yeast), plant cells, animal cells, mammalian cells and human cells.

The terms “operative linkage” and “operatively linked” are used with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. An operatively linked transcriptional regulatory sequence is generally joined in cis with a coding sequence, but need not be directly adjacent to it. For example, an enhancer can constitute a transcriptional regulatory sequence that is operatively-linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP DNA-binding domain is fused to a transcriptional activation domain (or functional fragment thereof), the ZFP DNA-binding domain and the transcriptional activation domain (or functional fragment thereof) are in operative linkage if, in the fusion polypeptide, the ZFP DNA-binding domain portion is able to bind its target site and/or its binding site, while the transcriptional activation domain (or functional fragment thereof) is able to activate transcription.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide analogues or substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

The term “recombinant,” when used with reference to a cell, indicates that the cell replicates an exogenous nucleic acid, or expresses a peptide or protein encoded by an exogenous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, that has control elements that are capable of effecting expression of a structural gene that is operatively linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding a desired polypeptide) and a promoter. Additional factors necessary or helpful in effecting expression can also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The term “naturally occurring,” as applied to an object, means that the object can be found in nature.

The terms “polypeptide,” “peptide” and “protein” are used interchangeably to refer to a polymer of amino acid residues. The term also applies to amino acid polymers in which one or more amino acids are chemical analogues of a corresponding naturally-occurring amino acids.

A “subsequence” or “segment” when used in reference to a nucleic acid or polypeptide refers to a sequence of nucleotides or amino acids that comprise a part of a longer sequence of nucleotides or amino acids (e.g., a polypeptide), respectively.

The term “antibody” as used herein includes antibodies obtained from both polyclonal- and monoclonal preparations, as well as, the following: (i) hybrid (chimeric) antibody molecules (see, for example, Winter et al. (1991) Nature 349:293-299; and U.S. Pat. No. 4,816,567); (ii) F(ab′)2 and F(ab) fragments; (iii) Fv molecules (noncovalent heterodimers, see, for example, Inbar et al. (1972) Proc. Natl. Acad. Sci. USA 69:2659-2662; and Ehrlich et al. (1980) Biochem 19:4091-4096); (iv) single-chain Fv molecules (sFv) (see, for example, Huston et al. (1988) Proc. Natl. Acad. Sci. USA 85:5879-5883); (v) dimeric and trimeric antibody fragment constructs; (vi) humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); (vii) Mini-antibodies or minibodies (i.e., sFv polypeptide chains that include oligomerization domains at their C-termini, separated from the sFv by a hinge region; see, e.g., Pack et al. (1992) Biochem 31:1579-1584; Cumber et al. (1992) J. Immunology 149B:120-126); and, (vii) any functional fragments obtained from such molecules, wherein such fragments retain specific-binding properties of the parent antibody molecule.

“Specific binding” between an antibody or other binding agent and an antigen, or between two binding partners, means that the dissociation constant for the interaction is less than 10⁻⁶ M. Preferred antibody/antigen or binding partner complexes have a dissociation constant of less than about 10⁻⁷ M, and preferably 10⁻⁸ M to 10⁻⁹ M or 10⁻¹⁰ M or lower.

Modulation of Gene Expression Using Insulator Domains

A. Insulator Domains

Insulator elements are special, cis-acting, chromosomal regions that serve as boundaries to prevent the transmission of chromatin structural features associated with repressive or active domains (Chung et al., supra). Insulator elements are typically located at the junctions between the decondensed chromatin of a transcriptionally active gene and the adjacent condensed chromatin. Further, certain insulator elements have been shown to play a role in establishing active or inactive chromatin structures. Insulator activity correlates with alterations in DNA accessibility to restriction enzymes caused by changes in nucleosome positioning (Gadula et al., (1996) PNAS USA 93:9378-9383). Further, insulator elements have also been shown to silence specific genes when positioned between an enhancer and a promoter of a target gene or in X-inactivation. (See, e.g., Wolffe, CHROMATIN STRUCTURE AND FUNCTION, Third edition, Academic Press, San Diego, 1998).

Trans-acting proteins that are involved in insulator functions have also been identified. Many of these insulator proteins include one or more DNA binding domains that specifically recognize and bind to known insulator elements. For example, the highly conserved zinc-finger protein, CTCF, is a candidate tumor suppressor protein that binds to highly divergent DNA sequences. One zinc-finger cluster of CTCF has been shown to silence transcription in all cell types tested and bind directly to the co-repressor SIN3A. (Golovnin et al. (1999) Mol Cell Biol. 19:3443-3456).

However, prior to the present disclosure, the functions of insulator proteins have been studied only in relation to natural binding sites and it has not been demonstrated that these proteins can be used to modulate expression of specific targeted genes. For example, it was not clear what role, if any, methylation of DNA played in insulation-related effects mediated by insulator proteins. Described herein is the identification of novel insulator elements in differentially methylated domains of the mammalian Igf2-H19 locus. Additionally described is the novel finding that the insulator protein CTCF functions to prevent enhancer blocking necessary for gene silencing and that the binding of the insulator protein is methylation sensitive. These findings allow the development and use of one or more of the functional domains of insulator proteins to modulate gene expression, by, for example, blocking the ability of an enhancer to activate a gene, or preventing silencing of genes associated with methylated regulatory regions. Further, these insulator domains may or may not directly bind to DNA.

Accordingly, in preferred embodiments, the fusion molecules described herein comprises a domain of an insulator polypeptide that is involved in modulation of gene expression, for example by silencing expression of a gene or by activating expression. Thus, a suitable insulator domain-containing composition can comprise one of its constituent proteins or a functional fragment thereof. Repression of a gene of interest can occur, for example, by employing a fusion of an insulator domain that interferes with enhancer function and a DNA binding domain which targets the gene of interest. Similarly, activation of a gene of interest can occur by employing a fusion of an insulator domain that prevents silencing (e.g., via the position effect) and a DNA binding domain which targets the gene of interest. In particular, transgenes or other exogenous sequences which have been integrated into a host genome rarely provide sustained expression of their gene product, often due to propagation of repressive effects from adjacent cellular chromatin. The methods and compositions described herein overcome these problems by allowing targeted regulation of both naturally situated and exogenous sequences.

Insulator domains can be isolated from known insulator proteins or synthesized as described herein. Preferably, the insulator domains or functional fragments thereof are derived from known insulator binding proteins including, for example, CTCF, the Drosophila suppressor of hair wing, su(Hw) (Wolffe (1994) Curr. Biol. 4:85-87), and polycomb group proteins, such as HPC2, RING1, suppressor of zeste (Su(z)₂), mod(mdg4) and the GAGA-binding Tr1 protein. See, for example, Bell et al. (1999) supra, and references cited therein, for a description of insulators and insulator binding proteins from which insulator domains can be obtained. See also van der Vlag et al (2000) J. Biol. Chem. 275:697-704 and references cited therein.

Additional insulator binding proteins comprising insulator domains can be obtained by one of skill in the art using established methods. Any protein capable of binding to an insulator sequence (see e.g., Bell et al. (1999) supra) can be used in the methods and compositions disclosed herein. Tests for the ability of a protein to bind to a specific DNA sequence are well-known to those of skill in the art and include, for example, electrophoretic mobility shift, nuclease and chemical footprinting, filter binding and chromatin immunoprecipitation. Accordingly, it is within the skill of the art to identify insulator binding proteins in addition to those disclosed herein.

B. DNA-Binding Domains

In certain embodiments, the compositions and methods disclosed herein involve fusions between a DNA-binding domain and an insulator domain. A DNA-binding domain can comprise any molecular entity capable of sequence-specific binding to chromosomal DNA. Binding can be mediated by electrostatic interactions, hydrophobic interactions, or any other type of chemical interaction. Examples of moieties which can comprise part of a DNA-binding domain include, but are not limited to, minor groove binders, major groove binders, antibiotics, intercalating agents, peptides, polypeptides, oligonucleotides, and nucleic acids. An example of a DNA-binding nucleic acid is a triplex-forming oligonucleotide.

Minor groove binders include substances which, by virtue of their steric and/or electrostatic properties, interact preferentially with the minor groove of double-stranded nucleic acids. Certain minor groove binders exhibit a preference for particular sequence compositions. For instance, netropsin, distamycin and CC-1065 are examples of minor groove binders which bind specifically to AT-rich sequences, particularly runs of A or T. WO 96/32496.

Many antibiotics are known to exert their effects by binding to DNA. Binding of antibiotics to DNA is often sequence-specific or exhibits sequence preferences. Actinomycin, for instance, is a relatively GC-specific DNA binding agent.

In a preferred embodiment, a DNA-binding domain is a polypeptide. Certain peptide and polypeptide sequences bind to double-stranded DNA in a sequence-specific manner. For example, transcription factors participate in transcription initiation by RNA Polymerase II through sequence-specific interactions with DNA in the promoter and/or enhancer regions of genes. Defined regions within the polypeptide sequence of various transcription factors have been shown to be responsible for sequence-specific binding to DNA. See, for example, Pabo et al. (1992) Ann. Rev. Biochem. 61:1053-1095 and references cited therein. These regions include, but are not limited to, motifs known as leucine zippers, helix-loop-helix (HLH) domains, helix-turn-helix domains, zinc fingers, β-sheet motifs, steroid receptor motifs, bZIP domains, homeodomains, AT-hooks and others. The amino acid sequences of these motifs are known and, in some cases, amino acids that are critical for sequence specificity have been identified. Polypeptides involved in other process involving DNA, such as replication, recombination and repair, will also have regions involved in specific interactions with DNA. Peptide sequences involved in specific DNA recognition, such as those found in transcription factors, can be obtained through recombinant DNA cloning and expression techniques or by chemical synthesis, and can be attached to other components of a fusion molecule by methods known in the art.

In a more preferred embodiment, a DNA-binding domain comprises a zinc finger DNA-binding domain. See, for example, Miller et al. (1985) EMBO J. 4:1609-1614; Rhodes et al. (1993) Scientific American Feb.: 56-65; and Klug (1999) J. Mol. Biol. 293:215-218. In one embodiment, a target site for a zinc finger DNA-binding domain is identified according to site selection rules disclosed in co-owned WO 00/42219. ZFP DNA-binding domains are designed and/or selected to recognize a particular target site as described in co-owned WO 00/42219; WO 00/41566; and U.S. Ser. No. 09/444,241 filed Nov. 19, 1999 and 09/535,088 filed Mar. 23, 2000; as well as U.S. Pat. Nos. 5,789,538; 6,007,408; 6,013,453; 6,140,081 and 6,140,466; and PCT publications WO 95/19431, WO 98/54311, WO 00/23464 and WO 00/27878.

Certain DNA-binding domains are capable of binding to DNA that is packaged in nucleosomes. See, for example, Cordingley et al. (1987) Cell 48:261-270; Pina et. al. (1990) Cell 60:719-731; and Cirillo et al. (1998) EMBO J. 17:244-254. Certain ZFP-containing proteins such as, for example, members of the nuclear hormone receptor superfamily, are capable of binding DNA sequences packaged into chromatin. These include, but are not limited to, the glucocorticoid receptor and the thyroid hormone receptor. Archer et al. (1992) Science 255:1573-1576; Wong et al. (1997) EMBO J. 16:7130-7145. Other DNA-binding domains, including certain ZFP-containing binding domains, require more accessible DNA for binding. In the latter case, the binding specificity of the DNA-binding domain can be determined by identifying accessible regions in the cellular chromatin. Accessible regions can be determined as described in co-owned U.S. Patent Application Ser. No. 60/228,556. A DNA-binding domain is then designed and/or selected to bind to a target site within the accessible region.

C. Fusion Molecules

The showing that insulator binding proteins contain domains involved in facilitating activation and repression of transcription by, for example, interfering with enhancer function, allows for the design of fusion molecules which facilitate regulation of gene expression. Thus, in certain embodiments, the compositions and methods disclosed herein involve fusions between a DNA-binding domain and an insulator domain or functional fragment thereof, as described supra, or a polynucleotide encoding such a fusion. In such a fusion molecule, an insulator domain is brought into proximity with a sequence in a gene that is bound by the DNA-binding domain. The transcriptional regulatory function of the insulator is then able to act on the gene, by, for example, modulating the ability of an enhancer to exert its function on the gene.

In additional embodiments, targeted remodeling of chromatin, as disclosed in co-owned U.S. patent application entitled “Targeted Modification of Chromatin Structure,” can be used to generate one or more sites in cellular chromatin that are accessible to the binding of a insulator domain/DNA binding domain fusion molecule.

Fusion molecules are constructed by methods of cloning and biochemical conjugation that are well-known to those of skill in the art. Fusion molecules comprise a DNA-binding domain and a component of a insulator domain or a functional fragment thereof. In certain embodiments, fusion molecules comprise a DNA-binding domain, an insulator domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of an insulator domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, Ill.) Catalogue. Methods and compositions for making fusions between a minor groove binder and a polypeptide have been described. Mapp et al. (2000) Proc. Natl. Acad. Sci. USA 97:3930-3935.

The fusion molecules disclosed herein comprise a DNA-binding domain which binds to a target site. In certain embodiments, the target site is present in an accessible region of cellular chromatin. Accessible regions can be determined as described in co-owned U.S. Patent Application Ser. No. 60/228,556. If the target site is not present in an accessible region of cellular chromatin, one or more accessible regions can be generated as described in co-owned U.S. patent application entitled “Targeted Modification of Chromatin Structure.” In additional embodiments, the DNA-binding domain of a fusion molecule is capable of binding to cellular chromatin regardless of whether its target site is in an accessible region or not. For example, such DNA-binding domains are capable of binding to linker DNA and/or nucleosomal DNA. Examples of this type of “pioneer” DNA binding domain are found in certain steroid receptor and in hepatocyte nuclear factor 3 (HNF3). Cordingley et al. (1987) Cell 48:261-270; Pina et al. (1990) Cell 60:719-731; and Cirillo et al. (1998) EMBO J. 17:244-254.

Methods of gene regulation using an insulator domain, targeted to a specific sequence by virtue of a fused DNA binding domain, can achieve modulation of gene expression. Modulation of gene expression can be in the form of increased expression (e.g., sustaining expression of an integrated transgene) or repression (e.g., repressing expression of exogenous genes, for example, when the target gene resides in a pathological infecting microorganism or in an endogenous gene of the subject, such as an oncogene or a viral receptor, that contributes to a disease state). As described supra, repression of a specific target gene can be achieved by using a fusion molecule comprising an insulator domain (or functional fragment thereof) and a DNA-binding domain, for interfering with enhancer function by using a specific DNA binding domain to target the insulator domain between an enhancer and promoter.

Alternatively, modulation can be in the form of activation, if activation of a gene (e.g., a tumor suppressor gene or a transgene) can ameliorate a disease state. In this case, cellular chromatin is contacted with a fusion molecule comprising an insulator domain and a DNA-binding domain, wherein the DNA-binding domain is specific for the target gene. The insulator domain portion of the fusion molecule enables sustained expression of the target gene, for example by preventing a “position effect” (e.g. by preventing context-dependent repression of a gene) by, for example, interfering with binding of trans acting factors and/or by itself recruiting additional factors that overcome the repressive environment of the target gene. These embodiments are particularly suitable for the activation of transgenes and for the activation of genes whose expression has been silenced during development, for example by genomic imprinting.

For such applications, the fusion molecule can be formulated with a pharmaceutically acceptable carrier, as is known to those of skill in the art. See, for example, Remington's Pharmaceutical Sciences, 17^{th} ed., 1985; and co-owned WO 00/42219.

Polynucleotide and Polypeptide Delivery

The compositions described herein can be provided to the target cell in vitro or in vivo. In addition, the compositions can be provided as polypeptides, polynucleotides or combination thereof.

A. Delivery of Polynucleotides

In certain embodiments, the compositions are provided as one or more polynucleotides. Further, as noted above, an insulator domain-containing composition can be designed as a fusion between a polypeptide DNA-binding domain and an insulator domain, that is encoded by a fusion nucleic acid. In both fusion and non-fusion cases, the nucleic acid can be cloned into intermediate vectors for transformation into prokaryotic or eukaryotic cells for replication and/or expression. Intermediate vectors for storage or manipulation of the nucleic acid or production of protein can be prokaryotic vectors, (e.g., plasmids), shuttle vectors, insect vectors, or viral vectors for example. An insulator domain-containing nucleic acid can also cloned into an expression vector, for administration to a bacterial cell, fungal cell, protozoal cell, plant cell, or animal cell, preferably a mammalian cell, more preferably a human cell.

To obtain expression of a cloned nucleic acid, it is typically subcloned into an expression vector that contains a promoter to direct transcription. Suitable bacterial and eukaryotic promoters are well known in the art and described, e.g., in Sambrook et al., supra; Ausubel et al., supra; and Kriegler, Gene Transfer and Expression: A Laboratory Manual (1990). Bacterial expression systems are available in, e.g., E. coli, Bacillus sp., and Salmonella. Palva et al. (1983) Gene 22:229-235. Kits for such expression systems are commercially available. Eukaryotic expression systems for mammalian cells, yeast, and insect cells are well known in the art and are also commercially available, for example, from Invitrogen, Carlsbad, Calif. and Clontech, Palo Alto, Calif.

The promoter used to direct expression of the nucleic acid of choice depends on the particular application. For example, a strong constitutive promoter is typically used for expression and purification. In contrast, when a protein is to be used in vivo, either a constitutive or an inducible promoter is used, depending on the particular use of the protein. In addition, a weak promoter can be used, such as HSV TK or a promoter having similar activity. The promoter typically can also include elements that are responsive to transactivation, e.g., hypoxia response elements, Gal4 response elements, lac repressor response element, and small molecule control systems such as tet-regulated systems and the RU-486 system. See, e.g., Gossen et al. (1992) Proc. Natl. Acad. Sci. USA 89:5547-5551; Oligino et al. (1998) Gene Ther. 5:491-496; Wang et al. (1997) Gene Ther. 4:432-441; Neering et al. (1996) Blood 88:1147-1155; and Rendahl et al. (1998) Nat. Biotechnol. 16:757-761.

In addition to a promoter, an expression vector typically contains a transcription unit or expression cassette that contains additional elements required for the expression of the nucleic acid in host cells, either prokaryotic or eukaryotic. A typical expression cassette thus contains a promoter operably linked, e.g., to the nucleic acid sequence, and signals required, e.g., for efficient polyadenylation of the transcript, transcriptional termination, ribosome binding, and/or translation termination. Additional elements of the cassette may include, e.g., enhancers, and heterologous spliced intronic signals.

The particular expression vector used to transport the genetic information into the cell is selected with regard to the intended use of the resulting insulator polypeptide, e.g., expression in plants, animals, bacteria, fungi, protozoa etc. Standard bacterial expression vectors include plasmids such as pBR322, pBR322-based plasmids, pSKF, pET23D, and commercially available fusion expression systems such as GST and LacZ. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High-yield expression systems are also suitable, such as baculovirus vectors in insect cells, with a nucleic acid sequence coding for an insulator domain under the transcriptional control of the polyhedrin promoter or any other strong baculovirus promoter.

Elements that are typically included in expression vectors also include a replicon that functions in E. coli (or in the prokaryotic host, if other than E. coli), a selective marker, e.g., a gene encoding antibiotic resistance, to permit selection of bacteria that harbor recombinant plasmids, and unique restriction sites in nonessential regions of the vector to allow insertion of recombinant sequences.

Standard transfection methods can be used to produce bacterial, mammalian, yeast, insect, or other cell lines that express large quantities of insulator domain proteins, which can be purified, if desired, using standard techniques. See, e.g., Colley et al. (1989) J. Biol. Chem. 264:17619-17622; and Guide to Protein Purification, in Methods in Enzymology, vol. 182 (Deutscher, ed.) 1990. Transformation of eukaryotic and prokaryotic cells are performed according to standard techniques. See, e.g., Morrison (1977) J. Bacteriol. 132:349-351; Clark-Curtiss et al. (1983) in Methods in Enzymology 101:347-362 (Wu et al., eds).

Any procedure for introducing foreign nucleotide sequences into host cells can be used. These include, but are not limited to, the use of calcium phosphate transfection, DEAE-dextran-mediated transfection, polybrene, protoplast fusion, electroporation, lipid-mediated delivery (e.g., liposomes), microinjection, particle bombardment, introduction of naked DNA, plasmid vectors, viral vectors (both episomal and integrative) and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

Conventional viral and non-viral based gene transfer methods can be used to introduce nucleic acids into mammalian cells or target tissues. Such methods can be used to administer nucleic acids encoding reprogramming polypeptides to cells in vitro. Preferably, nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For reviews of gene therapy procedures, see, for example, Anderson (1992) Science 256:808-813; Nabel et al. (1993) Trends Biotechnol. 11:211-217; Mitani et al. (1993) Trends Biotechnol. 11:162-166; Dillon (1993) Trends Biotechnol. 11:167-175; Miller (1992) Nature 357:455-460; Van Brunt (1988) Biotechnology 6(10):1149-1154; Vigne (1995) Restorative Neurology and Neuroscience 8:35-36; Kremer et al. (1995) British Medical Bulletin 51(1):31-44; Haddada et al., in Current Topics in Microbiology and Immunology, Doerfler and Böhm (eds), 1995; and Yu et al. (1994) Gene Therapy 1:13-26.

Methods of non-viral delivery of nucleic acids include lipofection, microinjection, ballistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386; 4,946,787; and 4,897,355 and lipofection reagents are sold commercially (e.g., Transfectam™ and Lipofectin™). Cationic and neutral lipids that are suitable for efficient receptor-recognition lipofection of polynucleotides include those of Felgner, WO 91/17424 and WO 91/16024. Nucleic acid can be delivered to cells (ex vivo administration) or to target tissues (in vivo administration).

The preparation of lipid:nucleic acid complexes, including targeted liposomes such as immunolipid complexes, is well known to those of skill in the art. See, e.g., Crystal (1995) Science 270:404-410; Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; and U.S. Pat. Nos. 4,186,183; 4,217,344; 4,235,871; 4,261,975; 4,485,054; 4,501,728; 4,774,085; 4,837,028 and 4,946,787.

The use of RNA or DNA virus-based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, wherein the modified cells are administered to patients (ex vivo). Conventional viral based systems for the delivery of ZFPs include retroviral, lentiviral, poxyiral, adenoviral, adeno-associated viral, vesicular stomatitis viral and herpesviral vectors. Integration in the host genome is possible with certain viral vectors, including the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, allowing alteration and/or expansion of the potential target cell population. Lentiviral vectors are retroviral vector that are able to transduce or infect non-dividing cells and typically produce high viral titers. Selection of a retroviral gene transfer system would therefore depend on the target tissue. Retroviral vectors have a packaging capacity of up to 6-10 kb of foreign sequence and are comprised of cis-acting long terminal repeats (LTRs). The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof. Buchscher et al. (1992) J. Virol. 66:2731-2739; Johann et al. (1992) J. Virol. 66:1635-1640; Sommerfelt et al. (1990) Virol. 176:58-59; Wilson et al. (1989) J. Virol. 63:2374-2378; Miller et al. (1991) J. Virol. 65:2220-2224; and PCT/US94/05700).

Adeno-associated virus (AAV) vectors are also used to transduce cells with target nucleic acids, e.g., in the in vitro production of nucleic acids and peptides, and for in vivo and ex vivo gene therapy procedures. See, e.g., West et al. (1987) Virology 160:38-47; U.S. Pat. No. 4,797,368; WO 93/24641; Kotin (1994) Hum. Gene Ther. 5:793-801; and Muzyczka (1994) J. Clin. Invest. 94:1351. Construction of recombinant AAV vectors are described in a number of publications, including U.S. Pat. No. 5,173,414; Tratschin et al. (1985) Mol. Cell. Biol. 5:3251-3260; Tratschin, et al. (1984) Mol. Cell. Biol. 4:2072-2081; Hermonat et al. (1984) Proc. Natl. Acad. Sci. USA 81:6466-6470; and Samulski et al. (1989) J. Virol. 63:3822-3828.

Recombinant adeno-associated virus vectors based on the defective and nonpathogenic parvovirus adeno-associated virus type 2 (AAV-2) are a promising gene delivery system. Exemplary AAV vectors are derived from a plasmid containing the AAV 145 bp inverted terminal repeats flanking a transgene expression cassette. Efficient gene transfer and stable transgene delivery due to integration into the genomes of the transduced cell are key features for this vector system. Wagner et al. (1998) Lancet 351® (9117): 1702-3; and Kearns et al. (1996) Gene Ther. 9:748-55.

pLASN and MFG-S are examples are retroviral vectors that have been used in clinical trials. Dunbar et al. (1995) Blood 85:3048-305; Kohn et al. (1995) Nature Med. 1:1017-102; Malech et al. (1997) Proc. Natl. Acad. Sci. USA 94:12133-12138. PA317/pLASN was the first therapeutic vector used in a gene therapy trial. (Blaese et al. (1995) Science 270:475-480. Transduction efficiencies of 50% or greater have been observed for MFG-S packaged vectors. Ellem et al. (1997) Immunol Immunother. 44(1):10-20; Dranoff et al. (1997) Hum. Gene Ther. 1:111-2.

In applications for which transient expression is preferred, adenoviral-based systems are useful. Adenoviral based vectors are capable of very high transduction efficiency in many cell types and are capable of infecting, and hence delivering nucleic acid to, both dividing and non-dividing cells. With such vectors, high titers and levels of expression have been obtained. Adenovirus vectors can be produced in large quantities in a relatively simple system.

Replication-deficient recombinant adenovirus (Ad) vectors can be produced at high titer and they readily infect a number of different cell types. Most adenovirus vectors are engineered such that a transgene replaces the Ad E1a, E1b, and/or E3 genes; the replication defector vector is propagated in human 293 cells that supply the required E1 functions in trans. Ad vectors can transduce multiple types of tissues in vivo, including non-dividing, differentiated cells such as those found in the liver, kidney and muscle. Conventional Ad vectors have a large carrying capacity for inserted DNA. An example of the use of an Ad vector in a clinical trial involved polynucleotide therapy for antitumor immunization with intramuscular injection. Sterman et al. (1998) Hum. Gene Ther. 7:1083-1089. Additional examples of the use of adenovirus vectors for gene transfer in clinical trials include Rosenecker et al. (1996) Infection 24:5-10; Sterman et al., supra; Welsh et al. (1995) Hum. Gene Ther. 2:205-218; Alvarez et al. (1997) Hum. Gene Ther. 5:597-613; and Topf et al. (1998) Gene Ther. 5:507-513.

Packaging cells are used to form virus particles that are capable of infecting a host cell. Such cells include 293 cells, which package adenovirus, and Ψ2 cells or PA317 cells, which package retroviruses. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. Missing viral functions are supplied in trans, if necessary, by the packaging cell line. For example, AAV vectors used in gene therapy typically only possess ITR sequences from the AAV genome, which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment, which preferentially inactivates adenoviruses.

In many gene therapy applications, it is desirable that the gene therapy vector be delivered with a high degree of specificity to a particular tissue type. A viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) Proc. Natl. Acad. Sci. USA 92:9747-9751 reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., F_{ab} or Fᵥ) having specific binding affinity for virtually any chosen cellular receptor. Although the above description applies primarily to viral vectors, the same principles can be applied to non-viral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described infra. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art. See, e.g., Freshney et al., Culture of Animal Cells, A Manual of Basic Technique, 3rd ed., 1994, and references cited therein, for a discussion of isolation and culture of cells from patients.

In one embodiment, hematopoietic stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ stem cells in vitro into clinically important immune cell types using cytokines such a GM-CSF, IFN-γ and TNF-α are known. Inaba et al. (1992) J. Exp. Med. 176:1693-1702.

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+(panB cells), GR-1 (granulocytes), and lad (differentiated antigen presenting cells). See Inaba et al., supra.

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions described herein. See, e.g., Remington's Pharmaceutical Sciences, 17th ed., 1989.

B. Delivery of Polypeptides

In other embodiments, fusion proteins are administered directly to target cells. In certain in vitro situations, the target cells are cultured in a medium containing insulator domain polypeptides (or functional fragments thereof) fused to a DNA binding domain.

An important factor in the administration of polypeptide compounds is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins, lipids and other compounds, which have the ability to translocate polypeptides across a cell membrane, have been described.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58. Prochiantz (1996) Curr. Opin. Neurobiol. 6:629-634. Another subsequence, the h (hydrophobic) domain of signal peptides, was found to have similar cell membrane translocation characteristics. Lin et al. (1995) J. Biol. Chem. 270:14255-14258.

Examples of peptide sequences which can be linked to an insulator domain polypeptide for facilitating its uptake into cells include, but are not limited to: an 11 amino acid peptide of the tat protein of HIV; a 20 residue peptide sequence which corresponds to amino acids 84-103 of the p16 protein (see Fahraeus et al. (1996) Curr. Biol. 6:84); the third helix of the 60-amino acid long homeodomain of Antennapedia (Derossi et ah (1994) J. Biol. Chem. 269:10444); the h region of a signal peptide, such as the Kaposi fibroblast growth factor (K-FGF) h region (Lin et al., supra); and the VP22 translocation domain from HSV (Elliot et al. (1997) Cell 88:223-233). Other suitable chemical moieties that provide enhanced cellular uptake can also be linked, either covalently or non-covalently, to the insulator domain polypeptides.

Toxin molecules also have the ability to transport polypeptides across cell membranes. Often, such molecules (called “binary toxins”) are composed of at least two parts: a translocation or binding domain and a separate toxin domain. Typically, the translocation domain, which can optionally be a polypeptide, binds to a cellular receptor, facilitating transport of the toxin into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used to deliver peptides to the cell cytosol as internal or amino-terminal fusions. Arora et al. (1993) J. Biol. Chem. 268:3334-3341; Perelle et al. (1993) Infect. Immun. 61:5147-5156; Stenmark et al. (1991) J. Cell Biol. 113:1025-1032; Donnelly et al. (1993) Proc. Natl. Acad. Sci. USA 90:3530-3534; Carbonetti et al. (1995) Abstr. Annu. Meet. Am. Soc. Microbiol. 95:295; Sebo et al. (1995) Infect. Immun. 63:3851-3857; Klimpel et al. (1992) Proc. Natl. Acad. Sci. USA. 89:10277-10281; and Novak et al. (1992) J. Biol. Chem. 267:17186-17193.

Such subsequences can be used to translocate polypeptides, including the polypeptides as disclosed herein, across a cell membrane. This is accomplished, for example, by derivatizing the fusion polypeptide with one of these translocation sequences, or by forming an additional fusion of the translocation sequence with the fusion polypeptide. Optionally, a linker can be used to link the fusion polypeptide and the translocation sequence. Any suitable linker can be used, e.g., a peptide linker.

A suitable polypeptide can also be introduced into an animal cell, preferably a mammalian cell, via liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell.

The liposome fuses with the plasma membrane, thereby releasing the compound into the cytosol. Alternatively, the liposome is phagocytosed or taken up by the cell in a transport vesicle. Once in the endosome or phagosome, the liposome is either degraded or it fuses with the membrane of the transport vesicle and releases its contents.

In current methods of drug delivery via liposomes, the liposome ultimately becomes permeable and releases the encapsulated compound at the target tissue or cell. For systemic or tissue specific delivery, this can be accomplished, for example, in a passive manner wherein the liposome bilayer is degraded over time through the action of various agents in the body. Alternatively, active drug release involves using an agent to induce a permeability change in the liposome vesicle. Liposome membranes can be constructed so that they become destabilized when the environment becomes acidic near the liposome membrane. See, e.g., Proc. Natl. Acad. Sci. USA 84:7851 (1987); Biochemistry 28:908 (1989). When liposomes are endocytosed by a target cell, for example, they become destabilized and release their contents. This destabilization is termed fusogenesis. Dioleoylphosphatidylethanolamine (DOPE) is the basis of many “fusogenic” systems.

For use with the methods and compositions disclosed herein, liposomes typically comprise a fusion polypeptide as disclosed herein, a lipid component, e.g., a neutral and/or cationic lipid, and optionally include a receptor-recognition molecule such as an antibody that binds to a predetermined cell surface receptor or ligand (e.g., an antigen). A variety of methods are available for preparing liposomes as described in, e.g.; U.S. Pat. Nos. 4,186,183; 4,217,344; 4,235,871; 4,261,975; 4,485,054; 4,501,728; 4,774,085; 4,837,028; 4,235,871; 4,261,975; 4,485,054; 4,501,728; 4,774,085; 4,837,028; 4,946,787; PCT Publication No. WO 91/17424; Szoka et al. (1980) Ann. Rev. Biophys. Bioeng. 9:467; Deamer et al. (1976) Biochim. Biophys. Acta 443:629-634; Fraley, et al. (1979) Proc. Natl. Acad. Sci. USA 76:3348-3352; Hope et al. (1985) Biochim. Biophys. Acta 812:55-65; Mayer et al. (1986) Biochim. Biophys. Acta 858:161-168; Williams et al. (1988) Proc. Natl. Acad. Sci. USA 85:242-246; Liposomes, Ostro (ed.), 1983, Chapter 1); Hope et al. (1986) Chem. Phys. Lip. 40:89; Gregoriadis, Liposome Technology (1984) and Lasic, Liposomes: from Physics to Applications (1993). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments, it may be desirable to target a liposome using targeting moieties that are specific to a particular cell type, tissue, and the like. Targeting of liposomes using a variety of targeting moieties (e.g., ligands, receptors, and monoclonal antibodies) has been previously described. See, e.g., U.S. Pat. Nos. 4,957,773 and 4,603,044.

Examples of targeting moieties include monoclonal antibodies specific to antigens associated with neoplasms, such as prostate cancer specific antigen and MAGE. Tumors can also be diagnosed by detecting gene products resulting from the activation or over-expression of oncogenes, such as ras or c-erbB2. In addition, many tumors express antigens normally expressed by fetal tissue, such as the alphafetoprotein (AFP) and carcinoembryonic antigen (CEA). Sites of viral infection can be diagnosed using various viral antigens such as hepatitis B core and surface antigens (HBVc, HBVs) hepatitis C antigens, Epstein-Barr virus antigens, human immunodeficiency type-1 virus (HIV-1) and papilloma virus antigens. Inflammation can be detected using molecules specifically recognized by surface molecules which are expressed at sites of inflammation such as integrins (e.g., VCAM-1), selectin receptors (e.g., ELAM-1) and the like.

Standard methods for coupling targeting agents to liposomes are used. These methods generally involve the incorporation into liposomes of lipid components, e.g., phosphatidylethanolamine, which can be activated for attachment of targeting agents, or incorporation of derivatized lipophilic compounds, such as lipid derivatized bleomycin. Antibody targeted liposomes can be constructed using, for instance, liposomes which incorporate protein A. See Renneisen et al. (1990) J. Biol. Chem. 265:16337-16342 and Leonetti et al. (1990) Proc. Natl. Acad. Sci. USA 87:2448-2451.

Pharmaceutical Compositions and Administration

Insulator domains and DNA binding domain (e.g., a zinc finger protein (ZFP)) fusion molecules as disclosed herein, and expression vectors encoding these polypeptides, can be used in conjunction with various methods of gene therapy to facilitate the action of a therapeutic gene product. In such applications, an insulator domain-ZFP can be administered directly to a patient, e.g., to facilitate the modulation of gene expression and for therapeutic or prophylactic applications, for example, cancer (including tumors associated with Wilms' third tumor gene), ischemia, diabetic retinopathy, macular degeneration, rheumatoid arthritis, psoriasis, HIV infection, sickle cell anemia, Alzheimer's disease, muscular dystrophy, neurodegenerative diseases, vascular disease, cystic fibrosis, stroke, and the like. Examples of microorganisms whose inhibition can be facilitated through use of the methods and compositions disclosed herein include pathogenic bacteria, e.g. Chlamydia, Rickettsial bacteria, Mycobacteria, Staphylococci, Streptococci, Pneumococci, Meningococci and Conococci, Klebsiella, Proteus, Serratia, Pseudomonas, Legionella, Diphtheria, Salmonella, Bacilli (e.g., anthrax), Vibrio (e.g., cholera), Clostridium (e.g., tetanus, botulism), Yersinia (e.g., plague), Leptospirosis, and Borrellia (e.g., Lyme disease bacteria); infectious fungus, e.g., Aspergillus, Candida species; protozoa such as sporozoa (e.g., Plasmodia), rhizopods (e.g., Entamoeba) and flagellates (Trypanosoma, Leishmania, Trichomonas, Giardia, etc.); viruses, e.g., hepatitis (A, B, or C), herpes viruses (e.g., VZV, HSV-1, HHV-6, HSV-II, CMV, and EBV), HIV, Ebola, Marburg and related hemorrhagic fever-causing viruses, adenoviruses, influenza viruses, flaviviruses, echoviruses, rhinoviruses, coxsackie viruses, cornaviruses, respiratory syncytial viruses, mumps viruses, rotaviruses, measles viruses, rubella viruses, parvoviruses, vaccinia viruses, HTLV viruses, retroviruses, lentiviruses, dengue viruses, papillomaviruses, polioviruses, rabies viruses, and arboviral encephalitis viruses, etc.

Administration of therapeutically effective amounts of an insulator domain-DNA-binding domain polypeptide or a nucleic acid encoding these fusion polypeptides is by any of the routes normally used for introducing polypeptides or nucleic acids into ultimate contact with the tissue to be treated. The polypeptides or nucleic acids are administered in any suitable manner, preferably with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions. See, e.g., Remington's Pharmaceutical Sciences, 17^{th} ed. 1985.

Insulator domains and insulator domain fusion polypeptides or nucleic acids, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, orally, topically, intraperitoneally, intravesically or intrathecally. The formulations of compounds can be presented in unit-dose or multi-dose sealed containers, such as ampoules and vials. Injection solutions and suspensions can be prepared from sterile powders, granules, and tablets of the kind known to those of skill in the art.

Applications

The compositions and methods disclosed herein can be used to facilitate a number of processes involving transcriptional regulation. These processes include, but are not limited to, transcription, replication, recombination, repair, integration, maintenance of telomeres, processes involved in chromosome stability and disjunction, and maintenance and propagation of chromatin structures. Accordingly, the methods and compositions disclosed herein can be used to affect any of these processes, as well as any other process which can be influenced by insulator domain and insulator domain fusion molecules' effect on gene expression and DNA binding proteins.

In preferred embodiments, an insulator domain/DNA-binding domain fusion is used to achieve targeted repression of gene expression. Targeting is based upon the specificity of the DNA-binding domain. In another embodiment, an insulator domain/DNA-binding domain fusion is used to achieve reactivation of a developmentally-silenced gene or to achieve sustained activation of a transgene. The DNA-binding domain is often targeted to a region outside of the coding region of the gene and, in certain embodiments, is targeted to a region outside the regulatory region(s) of the gene. In these embodiments, additional molecules, exogenous and/or endogenous, can be used to facilitate repression or activation of gene expression. The additional molecules can also be fusion molecules, for example, fusions between a DNA-binding domain and a functional domain such as an activation or repression domain. See, for example, co-owned WO 00/41566.

Accordingly, expression of any gene in any organism can be modulated using the methods and compositions disclosed herein, including therapeutically relevant genes, genes of infecting microorganisms, viral genes, and genes whose expression is modulated in the process of target validation. Such genes include, but are not limited to, Wilms' third tumor gene (WT3), vascular endothelial growth factor (VEGF), VEGF receptors flt and flk, CCR-5, low density lipoprotein receptor (LDLR), estrogen receptor, HER-2/neu, BRCA-1, BRCA-2, phosphoenolpyruvate carboxykinase (PEPCK), CYP7, fibrinogen, apolipoprotein A (ApoA), apolipoprotein B (ApoB), renin, phosphoenolpyruvate carboxykinase (PEPCK), CYP7, fibrinogen, nuclear factor KB (NF-κB), inhibitor of NF-κB (I-κB), tumor necrosis factors (e.g., TNF-α, TNF-β), interleukin-1 (IL-1), FAS (CD95), FAS ligand (CD95L), atrial natriuretic factor, platelet-derived factor (PDF), amyloid precursor protein (APP), tyrosinase, tyrosine hydroxylase, β-aspartyl hydroxylase, alkaline phosphatase, calpains (e.g., CAPN10) neuronal pentraxin receptor, adriamycin response protein, apolipoprotein E (apoE), leptin, leptin receptor, UCP-1, IL-1, IL-1 receptor, IL-2, IL-3, IL-4, IL-5, IL-6, IL-12, IL-15, interleukin receptors, G-CSF, GM-CSF, colony stimulating factor, erythropoietin (EPO), platelet-derived growth factor (PDGF), PDGF receptor, fibroblast growth factor (FGF), FGF receptor, PAF, p16, p19, p53, Rb, p21, myc, myb, globin, dystrophin, eutrophin, cystic fibrosis transmembrane conductance regulator (CFTR), GNDF, nerve growth factor (NGF), NGF receptor, epidermal growth factor (EGF), EGF receptor, transforming growth factors (e.g., TGF-α, TGF-β), fibroblast growth factor (FGF), interferons (e.g., IFN-α, IFN-β and IFN-γ), insulin-related growth factor-1 (IGF-1), angiostatin, ICAM-1, signal transducer and activator of transcription (STAT), androgen receptors, e-cadherin, cathepsins (e.g., cathepsin W), topoisomerase, telomerase, bcl, bcl-2, Bax, T Cell-specific tyrosine kinase (Zck), p38 mitogen-activated protein kinase, protein tyrosine phosphatase (hPTP), adenylate cyclase, guanylate cyclase, α7 neuronal nicotinic acetylcholine receptor, 5-hydroxytryptamine (serotonin)-2A receptor, transcription elongation factor-3 (TEF-3), phosphatidylcholine transferase, ftz, PTI-1, polygalacturonase, EPSP synthase, FAD2-1, Δ-9 desaturase, Δ-12 desaturase, Δ-15 desaturase, acetyl-Coenzyme A carboxylase, acyl-ACP thioesterase, ADP-glucose pyrophosphorylase, starch synthase, cellulose synthase, sucrose synthase, fatty acid hydroperoxide lyase, and peroxisome proliferator-activated receptors, such as PPAR-γ2.

Expression of human, mammalian, bacterial, fungal, protozoal, Archaeal, plant and viral genes can be modulated; viral genes include, but are not limited to, hepatitis virus genes such as, for example, HBV-C, HBV-S, HBV-X and HBV-P; and HIV genes such as, for example, tat and rev. Modulation of expression of genes encoding antigens of a pathogenic organism can be achieved using the disclosed methods and compositions.

Additional genes include those encoding cytokines, lymphokines, interleukins, growth factors, mitogenic factors, apoptotic factors, cytochromes, chemotactic factors, chemokine receptors (e.g., CCR-2, CCR-3, CCR-5, CXCR-4), phospholipases (e.g., phospholipase C), nuclear receptors, retinoid receptors, organellar receptors, hormones, hormone receptors, oncogenes, tumor suppressors, cyclins, cell cycle checkpoint proteins (e.g., Chk1, Chk2), senescence-associated genes, immunoglobulins, genes encoding heavy metal chelators, protein tyrosine kinases, protein tyrosine phosphatases, tumor necrosis factor receptor-associated factors (e.g., Traf-3, Traf-6), apolipoproteins, thrombic factors, vasoactive factors, neuroreceptors, cell surface receptors, G-proteins, G-protein-coupled receptors (e.g., substance K receptor, angiotensin receptor, α- and β-adrenergic receptors, serotonin receptors, and PAF receptor), muscarinic receptors, acetylcholine receptors, GABA receptors, glutamate receptors, dopamine receptors, adhesion proteins (e.g., CAMs, selectins, integrins and immunoglobulin superfamily members), ion channels, receptor-associated factors, hematopoietic factors, transcription factors, and molecules involved in signal transduction. Expression of disease-related genes, and/or of one or more genes specific to a particular tissue or cell type such as, for example, brain, muscle, heart, nervous system, circulatory system, reproductive system, genitourinary system, digestive system and respiratory system can also be modulated.

Thus, the methods and compositions disclosed herein can be used in processes such as, for example, therapeutic regulation of disease-related genes, engineering of cells for manufacture of protein pharmaceuticals, pharmaceutical discovery (including target discovery, target validation and engineering of cells for high throughput screening methods) and plant agriculture.

### EXAMPLES

The following examples are presented as illustrative of, but not limiting, the claimed subject matter.

### Example 1

### Materials and Methods

Mouse Strains and Tissues

M. m. musculus (M) (CZECH II, Jackson Laboratories) and M. m. domesticus (D) (NRMI strain) mice were used to create intra-specific F1 hybrid conceptuses. These were referred to as D×M or M×D conceptuses consistently, in the order mother-father. Fetuses were collected using natural matings, taking the date of vaginal plug formation as day 0.5 postcoitum. Fetal livers were collected at day 16.5 postcoitum.

Analysis of the In Vivo Interaction between CTCF and the H19 DMD

Fetal mouse liver cells were mechanically dispersed and formaldehyde-crosslinked, as described in Kuo et al. (1999) Methods 18:425-433. Following isolation of nuclei and sonication to shear the DNA, the CTCF-containing DNA-protein complexes were immunopurified using a CTCF antibody (Upstate Biotechnology, Lake Placid, N.Y.) and protein A 4 Fast Flow Sepharose beads (Pharmacia-Upjohn). The immunopurified DNA (the CTCF antibody was quantitatively recovered during the immunoprecipitation) was PCR-amplified using a ³²P-end labeled forward primer 5′-CGGGACTCCCAAAATCAACAAG-3′ (SEQ ID NO: 1) and an unlabeled reverse primer 5′-GCAATCCGTTTTAGGACTGC-3′ (SEQ ID NO: 2). PCR conditions were 1×94° C. for 5 min, 3×94° C. for 1 min, 1×57° C. for 1 min, 1×72° C. for 1 min, 24×(94° C. for 45 sec, 57° C. for 30 sec, 72° C. for 30 sec), and 1×72° C. for 5 min. The PCR products were phenol/chloroform-extracted, digested with BamHI and analyzed on non-denaturing 6% polyacrylamide gels. Dilution experiments showed that both parental alleles of the H19 differentially methylated domain (DMD) were quantitatively amplified using these conditions.

In Vitro Methylation

Purified fragments (5 μg per experiment) were methylated with 2 units/μg MSssI methyltransferase (New England BioLabs, Beverly, Mass.) in the presence of 180 μM S-Adenosyl methionine for 16 h at 37° C., using buffer conditions recommended by the manufacturer. Following termination of methylation reaction by heating at 65° C. for 15 min, the methylation status of plasmid constructs was analyzed by digesting with excess amounts of HhaI and BstUI overnight.

Point Mutations of the CTCF Cis Elements

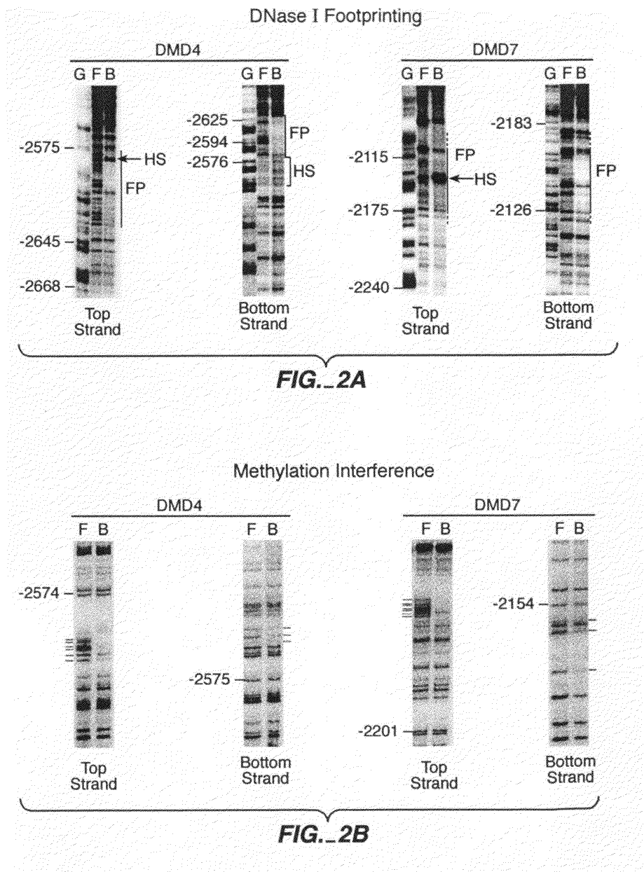

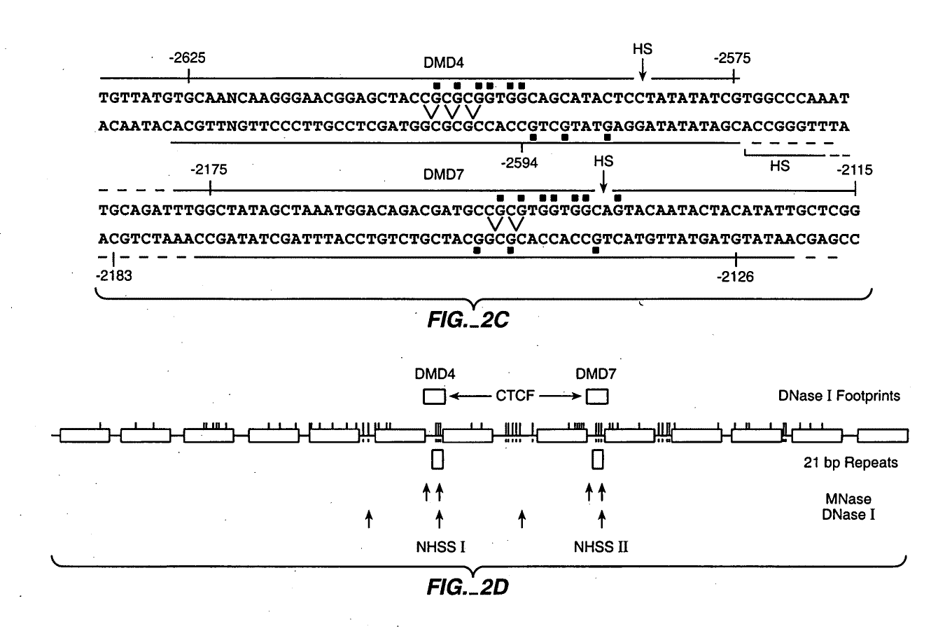

The QuikChange method (Stratagene) was used to destroy the CTCF recognition elements within the H19 DMD. Specifically, the sequence GTGG within the 21 bp repeat was converted to ATAT to generate the S1 and S2 mutants that correspond to the NHSS I and II (see FIG. 2), respectively. The S1 mutant was generated by using the following primers: forward—5′CGGAGCTACCGCGCGATATCAGCATACTCC-3′ (SEQ ID NO: 3); reverse—5′GGAGTATGCTGATATCGCGCGGTAGCTCCG-3′ (SEQ ID NO: 4). The S2 mutant was generated by using the following primers: forward -5′-GACGATGCCGCGTGATATCAGTACAATACTAC-3′ (SEQ ID NO: 5); reverse -5′-GTAGTATTGTACTGATATCACGCGGCATCGTC-3′ (SEQ ID NO: 6). The double mutants were generated by creating an S1 mutant on an S2 mutant background. The mutagenesis was performed using an intermediate cloning vector pCR2:1 (Invitrogen). The insertion of the mutagenized H19 5′-flanks into pREPH19 vectors was performed as described in Kanduri et al. (2000) Curr Biol 10:449-457. All the constructs were confirmed by sequencing and were subsequently prepared for transfection by propagation in the XL1 Blue strain of E. coli.

DNA-Protein Interaction Assays

DNase I footprinting, DMS interference, and gel-shift assays were carried out as described in Filippova et al. (1996) Mol Cell Biol 16:2802-2813.

Affinity Determinations

The BIACORE CM-5 chip (Biacore AB) was first coated with the affinity purified anti-amino-terminal CTCF region rabbit polyclonal antibodies (Upstate Biotechnology, Lake Placid, N.Y.) on the experimental well and with the protein-G purified rabbit non-immune IgG fraction on the control well by the amino-coupling procedure according to manufacturer's instructions. Then in vitro-translated CTCF diluted 1:5 with the running buffer RB (25 mM HEPES pH 7.4, 100 mM KCl, 2 mM MgCl₂, 1 mM DTT, 0.1 mM ZnSO₄, 2.5% CHAPS, 1 μg/ml poly(dI-dC), and 10 μg/ml BSA) was run through both wells. On average, in three independent experiments, about 140-150 RU remained bound to the experimental well after extensive washing. Gel-purified DMD4 and DMD7 control or methylated with SssI methylase DNA fragments at concentrations from 10 nM to 100 nM were run through the wells in the RB. Next, wells were regenerated by washing off CTCF-DNA complexes from the immobilized antibodies by passing 60 μl of 100 mM-glycine pH 2.5. This cycle was repeated for each measurement. Binding of DNA to CTCF was analyzed using the Biacore software supplied by the manufacturer.

Enhancer-Blocking Analyses

The JEG-3 cell line was maintained in MEM (Gibco BRL) as has been described by Franklin et al. (1996) Oncogene 11:1173-1184. The transfection of plasmid DNAs into these cells followed previously published protocols (e.g., Awad et al. (1999) J. Biol Chem 274:27082-27098). The activity of the promoter of the H19 reporter gene was determined by RNase protection, as described in Walsh et al. (1994) Mech Dev 46:55-62. Quantification of individual protected fragments was carried out in Fuji Bas 1500 Phosphormager. The H19 expression signals were corrected both with respect to internal control (PDGFB signal) and episome copy number, which was determined by Southern blot analysis of ApaI-restricted DNA as described by Walsh et al., supra.

### Example 2

### Identification of a CTCF Binding Sites in H19 Locus

The chromatin structure of the H19 DMD displays several unusual features, including multiple nuclease hypersensitive sites (NHSSs) that map to linker regions flanked by positioned nucleosomes in the maternally-inherited allele. The most prominent of these nuclease hypersensitive sites map to a 21 bp element that is repeated several times in both the mouse H19 DMD and in its human counterpart. When the nucleotide sequence of this 21 bp repeat was compared to functional cis elements within the β-globin insulator, similarity of the 21 bp repeats to a CTCF binding site in the globin insulator was observed.

CTCF is an evolutionarily-conserved, ubiquitously-expressed protein, containing 11 zinc fingers, that is capable of binding to a wide variety of target sites with different sequences by utilizing different subsets of its zinc fingers. Different types of CTCF target sites mediate various CTCF-mediated functions, including promoter repression, promoter activation and hormone-responsive repression of gene expression. Lobanenkov et al. (1990) Oncogene 5:1743-1753; Filippova et al. (1996) Mol. Cell. Biol. 16:2802-2813; Vostrov et al. (1997) J. Biol. Chem. 272:33,353-33,359; Yang et al. (1999) J. Neurochem. 73:2286-2298; Burcin et al. (1997) Mol. Cell. Biol. 17:1281-1288; Awad et al. (1999) J. Biol. Chem. 274:27,092-27,098. A number of CTCF binding sites have been reported to comprise the enhancer blocking elements of chromatin insulators in vertebrates. Bell et al. (1999) Cell 98:387-396.

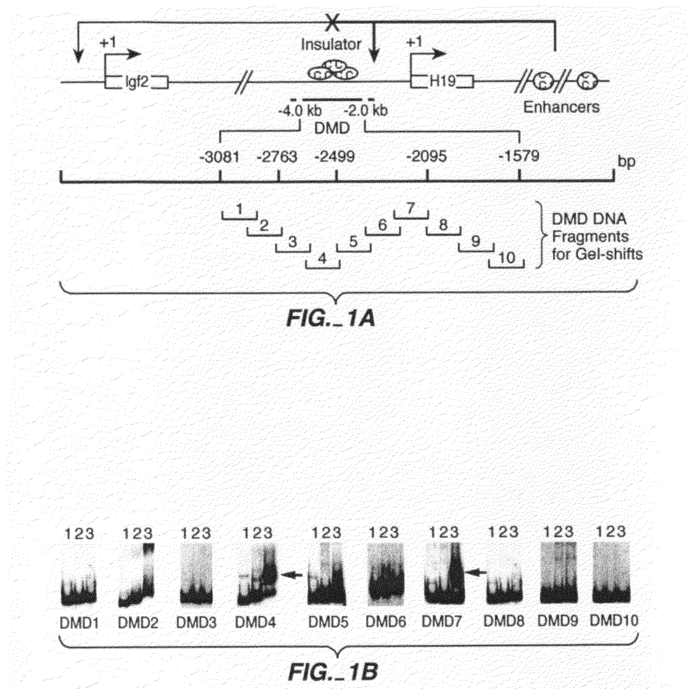

To directly test a potential link between CTCF and the differentially methylated domain (DMD) of the 5′ flanking region of H19, systematic CTCF binding analyses of the H19 5′ non-coding region from positions −1579 to −3081 (relative to the H19 transcription start site) were carried out, using gel mobility super shifting assays, essentially as described in Filippova et al. (1996) Mol. Cell. Biol. 16:2802-2813. FIG. 1A is a schematic depicting DMD fragments used in the binding analysis and FIG. 1B shows the results, which indicate that two new CTCF-binding sites were identified, termed DMD4 and DMD7. Gel mobility super-shifting experiments with CTCF antibodies showed that both DMD4 and DMD7 CTCF-target sequences specifically interacted with the endogenous CTCF protein present in nuclear extracts. Thus, CTCF represents the major nuclear protein binding to these sequences.

### Example 3

### Characterization of DMD4 and DMD7 CTCF-Binding Sequences

DNase 1 footprinting and DMS-methylation interference methods, as previously described in Lobanenkov et al. (1990) Oncogene 5:1743-1753; Klenova et al. (1993) Mol. Cell. Biol. 13:7612-7624 and Filippova et al. (1996) Mol. Cell. Biol. 17:1281-1288, were used to further characterize the binding of the CTCF ZF domain to DMD4 and DMD7. Each 5′-end-labeled strand of the DMD4 and DMD7 DNA fragments was used in these assays in order to define exactly which sequences were occupied by CTCF and to identify guanines within these sequences which could not be modified without losing CTCF binding. DNAse I footprinting analyses are shown in FIG. 2A. Methylation interference assays are shown in FIG. 2B.

The results shown in FIGS. 2A through 2D indicate that the binding sites for CTCF within the DMD4 and DMD7 fragments corresponded precisely with the previously-determined sites of nuclease hypersensitive in chromatin (NHSSI and NHSSII), respectively. Further, in each recognition sequence, CTCF protected approximately 60 bp of both DNA strands from nuclease attack. In addition, inside of each binding site, DNA-bound CTCF induced DNase 1 hypersensitive subsites on the top GC-rich strand (marked as “HS” in the FIGS. 2A and C to distinguish them from the NHSSs in chromatin). Binding of CTCF is known to result in a severe bending of a target DNA sequences and there is also an allosteric effect of primary DNA sequence on the degree of DNA bending induced by CTCF binding at a given target site and the exact location of an HS is usually close to the center of CTCF-induced DNA bends (Arnold et al. (1996) Nucleic Acids Res. 24:2640-2547). In both DMD4 and DMD7, the identical CGCG(T/G)GGTGGCAG-core sequence (SEQ ID NO: 7) of the conserved 21 bp H19 DMD repeats provided major contact bases for recognition by CTCF. Finally, the DMD4 and DMD7 CTCF-recognition cores contained three and two CpGs, respectively, which are methylated in vivo on the paternal chromosome.

### Example 4

### Methylation of DMD4 and DMD7 Interferes with CTCF Binding

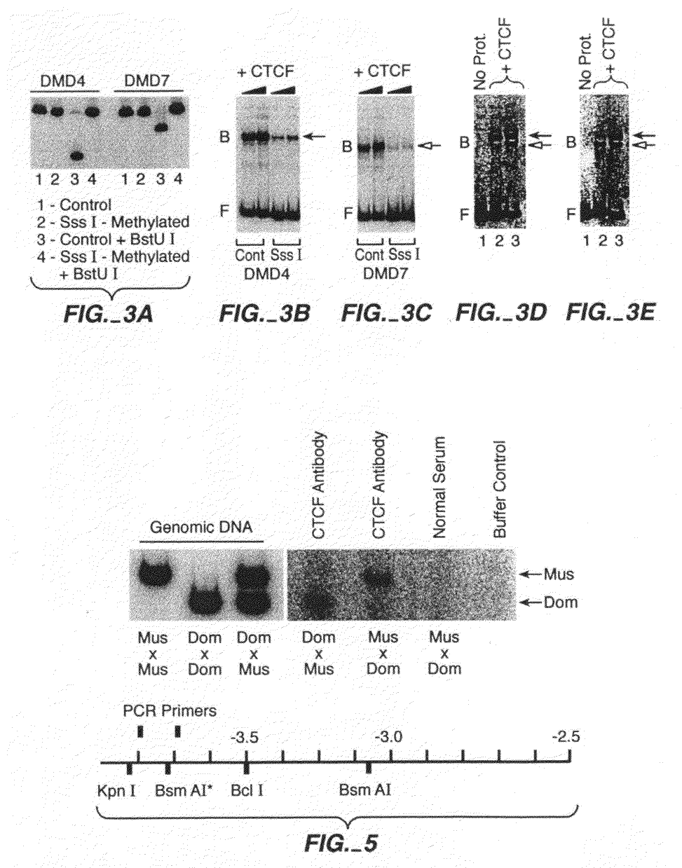

To test whether methylation of CpGs on the paternal chromosome would influences CTCF binding, the DMD4 and DMD7 fragments were modified with the SssI methylase. See Example 1. Complete methylation of the MSssI substrate CpG pairs within the CTCF-recognition motifs in the DMD4 and DMD7 fragments (FIG. 2C) was verified by resistance to BstUI digestion, as shown in FIG. 3A. Since these CpG pairs create the cutting sites for the methylation-sensitive restriction enzyme BstUI, methylation of these sites to completion results in resistance to BstUI digestion (FIG. 3A, lanes 4).

Methylated and unmethylated DMD4 and DMD7 fragments were compared for their ability to bind CTCF by electrophoretic mobility shift assays, and the results are shown in FIGS. 3B and 3C. Site-specific CpG methylation dramatically decreased CTCF binding to both the DMD4 (FIG. 3B) and DMD7 (FIG. 3C) sites. The differences in electrophoretic mobility of the DNA-CTCF complexes (formed with the two sites positioned at different distance from the ends of the DMD fragments) observed in these assays was due to a severe DNA bending induced by CTCF. Bell et al. (1999) Cell 98:387-396. This difference allowed a comparison between CTCF binding to the two fragments, methylated DMD7 plus control DMD4 and vice versa, mixed together at a 1:1 ratio. CTCF exhibited a marked preference for the unmethylated DMD sites (FIGS. 3D, 3E).

The effect of CpG-methylation on the affinity of CTCF binding to each DMD target was also quantitatively estimated, by utilizing surface plasmon resonance using the BIACORE X device. See Example 1. It appeared, quite unexpectedly, that the best-fit model for CTCF-DNA interaction was a two-stage reaction, with an intermediate conformational change resulting in formation of stable non-dissociating complexes with an apparent affinity constant in the range of 10¹¹ to 10¹³ M⁻¹. In contrast, CTCF binding to the methylated DMD4 and DMD7 sites was at least 1.000-fold lower in affinity (approximately 10⁸ M⁻¹), and no stable complexes with methylated probes were detected. CTCF affinity to the methylated DMDs was still high enough to detect some residual binding in gel shift experiments (FIG. 3). Taken together, these results demonstrate that the CpG methylation status of the CTCF binding site is a potent regulator of the interaction between CTCF and the H19 5′-flanking DMDs, with methylation inhibiting CTCF binding.

### Example 5

### Mutational Analysis of CTCF Binding Sites

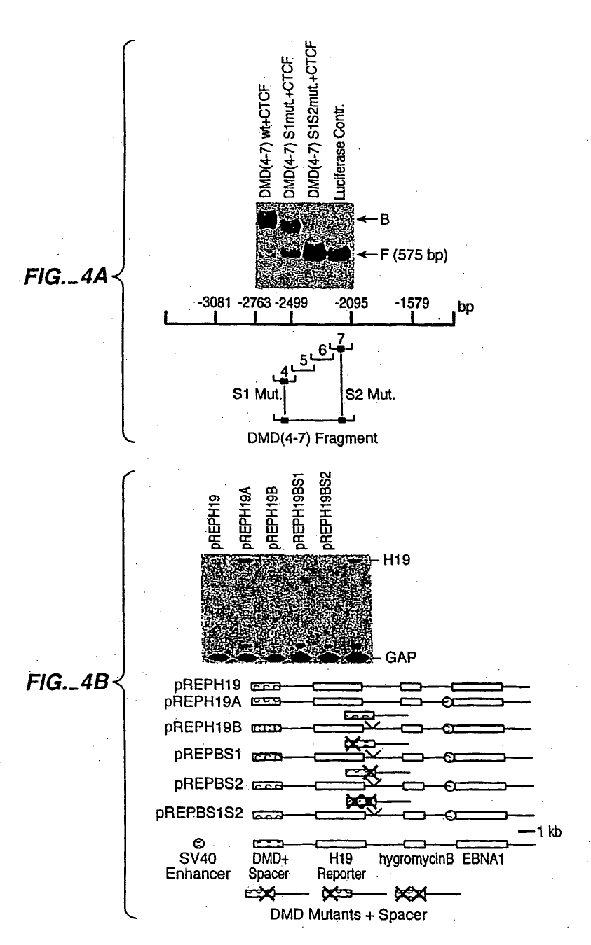

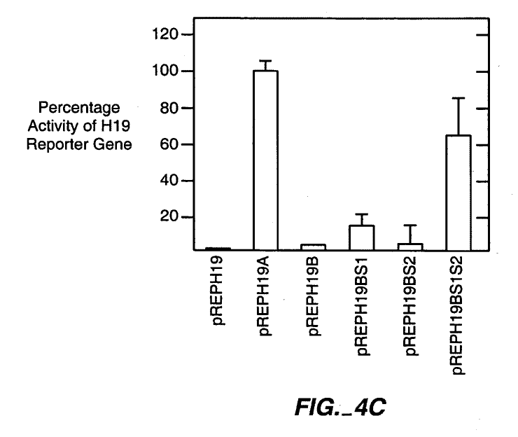

Chromatin-insulator-like activity appears to be a default function of different CTCF-binding sites when these are positioned between an enhancer and a promoter (Bell et al., supra). To examine whether the CTCF binding sites in the H19 DMD possess insulator activity, point mutations that eliminate CTCF interaction with the DMD4 and DMD7 sites were generated. Changing the sequence “GTGG” to “ATAT” in either of the CTCF binding sites (see FIG. 2C) blocked CTCF binding to its recognition sites in the H19 DMD, as examined by electrophoretic mobility shift analysis of a 575 bp fragment containing the DMD4 and DMD7 sites (FIG. 4A). These mutant sequences, which lack the ability to bind CTCF, were then used in an episomal-based assay for insulator function as described in Kanduri et al. (2000) Curr Biol. 10:449-457. This assay essentially determines the ability of either wild-type or mutant H19 DMDs to prevent the SV40 enhancer from activating the H19 promoter which drives expression of the reporter gene. The results of this analysis, shown in FIGS. 4B and 4C, indicated that targeted disruption of CTCF-DMD interaction at both sites counteracted most of the enhancer-blocking properties of the H19 5′-flanking DMD. Thus, inhibition of the binding of CTCF to its recognition sites in DMD4 and DMD7 results in loss of insulator function.

### Example 6

### Distribution of CTCF in Mouse Embryos

To ascertain if there is an in vivo link between CTCF and the H19 5′-flanking region, a chromatin immunopurification method (essentially as described in Kuo and Allis (1999) Methods 19:425-433) was utilized to analyze the distribution of CTCF in the chromatin of mouse fetuses. Formaldehyde-crosslinked chromatin of fetal livers was obtained from reciprocal M. musculus musculus×M. musculus domesticus intraspecific hybrid crosses, fragmented, and fragments immunoprecipitated using a CTCF polyclonal antibody. Following reversal of crosslink and removal of protein, immunoprecipitated DNA was analyzed by PCR amplification. The PCR assay allowed the discrimination of the parental alleles of the H19 5′-flank, by means of a polymorphic BsmAI restriction site situated towards the 5′-end of the differentially methylated domain of the H19 5′-flank (Kanduri et al, supra). Results are shown in FIG. 5. Only the maternally-inherited allele (the M. musculus musculus allele in the M×D cross) was specifically captured by the CTCF antibody (FIG. 5, right panel). When the reciprocal cross (D×M) was examined, the M. musculus domesticus allele was preferentially amplified. These results indicate that, in fetal liver, CTCF binds preferentially to the maternal allele of the H19 DMD. Given that the average length of the sonicated DNA fragments was between 2-3 kb, most, if not all, of the potential CTCF binding sites scattered within the DMD of the H19 5′-flank would likely have been detected in this assay. Therefore, CTCF-specific interaction with the H19 5′-flank is parent of origin-specific and corresponds with the in vitro binding results described above.

Thus, CTCF is both structurally and functionally an integral part of the H19 DMD chromatin conformation and is involved in maintaining and/or manifesting the repressed status of the maternal Igf2 allele in the soma. Furthermore, the parent of origin-dependent interaction of CTCF with the H19 insulator is determined, at least in part, by differential methylation of the maternal and paternal H19 alleles.

A more global function for CTCF in imprinting is suggested by the preponderance of sites, in the mammalian genome, having homology to known CTCF binding sites. Additional functions for CTCF are also possible. For example, the frequently observed loss of imprinting resulting in biallelic expression of Igf2 in Wilms' tumor may be related to the proposed function of CTCF as a tumor suppressor gene at chromosome segment 16q22, where the predicted third Wilms' tumor gene (WT3) is located. Tycko (1999) Genomic Imprinting in Cancer, in Genomic Imprinting: An Interdisciplinary Approach (Ohlsson, R. ed.) Vol. 25, pp. 133-170, Springer-Verlag, Berlin, Heidelberg, New York; Ohlsson et al. (1999) Cancer Res. 59:3889-3892; Filippova et al. (1998) Genes, Chromosomes, Cancer 22:26-36; Maw et al. (1992) Cancer Res. 52:3094-3098.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A method of modulating expression of a gene, the method comprising the step of contacting a region of DNA in cellular chromatin with a fusion polypeptide that binds to a binding site in cellular chromatin, wherein the fusion polypeptide comprises a DNA binding domain or functional fragment thereof and an insulator domain or functional fragment thereof.

2. The method of claim 1, wherein the DNA-bihding domain of the fusion polypeptide comprises a zinc finger DNA-binding domain.

3. The method of claim 1, wherein the insulator domain is derived from CTCF.

4. The method of claim 1, wherein the gene is in a plant cell.

5. The method of claim 1, wherein the gene is in an animal cell.

6. The method of claim 5, wherein the cell is a human cell.

7. The method of claim 1, Wherein modulation comprises repression of expression of the gene.

8. The method of claim 1, wherein the binding site is between an enhancer and a promoter further wherein binding of the fusion polypeptide interferes with the function of the enhancer.

9. The method of claim 1, wherein the modulation comprises preventing repression.

10. The method of claim 9, wherein the gene is a transgene.

11. The method of claim 1, wherein the modulation comprises activation of the gene.

12. The method of claim 11, wherein the gene is a transgene.

13. The method of claim 1, wherein the method further comprises the step of contacting the cell with a polynucleotide encoding the fusion polypeptide, wherein the fusion polypeptide is expressed in the cell.

14. The method of claim 1, wherein a plurality of fusion polypeptides are contacted with cellular chromatin, wherein each of the fusion polypeptides binds to a distinct binding site.

15. The method of claim 14, wherein at least one of the fusion polypeptides comprises a zinc finger DNA-binding domain.

16. The method of claim 14, wherein the expression of a plurality of genes is modulated.

17. The method of claim 14, wherein the cellular chromatin is in a plant cell.

18. The method of claim 14, wherein the cellular chromatin is in an animal cell.

19. The method of claim 18, wherein the cell is a human cell.

20. A method of altering the chromatin structure of a gene comprising the step of (a) contacting a region of DNA in cellular chromatin with a fusion polypeptide that binds to a binding site in cellular chromatin, wherein the fusion polypeptide comprises a DNA binding domain or functional fragment thereof and an insulator domain or functional fragment thereof.


SEQUENCE LISTING (7 sequences)

SEQ ID NO: 1 - DNA, 22 bp, Artificial
  forward primer
cgggactcccaaaatcaacaag

SEQ ID NO: 2 - DNA, 20 bp, Artificial
  reverse primer
gcaatccgttttaggactgc

SEQ ID NO: 3 - DNA, 30 bp, Artificial
  S1 mutant forward primer
cggagctaccgcgcgatatcagcatactcc

SEQ ID NO: 4 - DNA, 30 bp, Artificial
  S1 mutant reverse primer
ggagtatgctgatatcgcgcggtagctccg

SEQ ID NO: 5 - DNA, 32 bp, Artificial
  S2 mutant forward primer
gacgatgccgcgtgatatcagtacaatactac

SEQ ID NO: 6 - DNA, 32 bp, Artificial
  S2 mutant reverse primer
gtagtattgtactgatatcacgcggcatcgtc

SEQ ID NO: 7 - DNA, 13 bp, Artificial
  core sequence
cgcgkggtggcag